(19) United States
(12) Patent Application Publication
(10) Pub. No.: US 2006/0235419 A1
(43) Pub. Date: Oct. 19, 2006

(54) Medical instrument for performing microfracturing in a bone

(75) Inventors: Matthias Steinwachs, Freiburg (DE); Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(73) Assignee: Karl Storz GmbH & Co. KG

(21) Appl. No.: 11/367,207

(22) Filed: Mar. 3, 2006

(30) Foreign Application Priority Data
Mar. 3, 2005 (DE) 10 2005 010 989.6

(51) Int. Cl. A61F 5/00

(52) U.S. Cl. 606/86

(57) ABSTRACT

A medical instrument for performing microfracturing in a bone comprises a body, a plurality of points project in the distal direction.

### CROSS-REFERENCE TO RELATED APPLICATIONS

The present application claims priority of German patent application No. 10 2005 010 989.6 filed on Mar. 3, 2005.

### BACKGROUND OF THE INVENTION

The invention relates to a medical instrument for performing microfracturing in a bone.

Formation of microfractures in a bone, also referred to as “microfracturing”, is a technique for the repair of cartilage defects.

Instruments for performing microfractures in a bone are sold by the company Artrex GmbH, Munich, Germany, under the brand name “Chondro Pick”.

The articular cartilage in the area of the human knee joint differs in thickness according to the topography. In the area of the patella, it can reach a layer thickness of 7 to 8 mm. Since the articular cartilage does not have any direct vessel or nerve attachments, it is nourished mainly through diffusion from the synovial fluid of the intraarticular space. The cross-linking of various matrix components to form the cartilage ground substance permits mechanical damping and almost frictionless sliding of the articular surfaces. At the cellular level, there is a complex structure of cartilage cells (chondrocytes), collagen fibers and proteoglycans. The healthy cartilage in the area of the knee of a human adult can be charged during movement with forces that can amount to a multiple of the body's weight.

Damage to the articular cartilage represents a major problem in routine traumatology and orthopedics. The limited healing capacity of cartilage has long been known and is due essentially to the latter's particular structure and anatomy.

Damage to the articular surface, above all in the area of the load-bearing zones of the sliding surface of the joint, therefore entails increased risk of substantial joint damage in the sense of premature arthrosis.

For biological reconstruction of full-layer cartilage damage involving small and medium-sized defects, one of the techniques used is known as “microfracturing”.

In this technique, a pointed instrument is used to create a plurality of small openings, or microfractures, in the bone lying under the cartilage. These openings or microfractures extending as far as the bone marrow. Blood and bone marrow are able to pass through these microfractures to the surface of the bone in the area of the defect.

The underlying concept of this treatment method is that of generating a clot permeated with pluripotent stem cells from the bone marrow. Such a clot is also sometimes known as a “Super Clot”. The stem cells contained in this clot can differentiate to cartilage cells in the course of the healing process and form new cartilage tissue.

However, the presently used instruments (see, for example, US 2004/0147931 A1) have the disadvantage that each microfracture in the bone has to be formed individually. This is a very laborious process.

This technique also has the disadvantage that, in the joint spaces, which are often not readily accessible, it is difficult for the operating surgeon to achieve a uniform distribution or pattern of the microfractures and, consequently, a uniform distribution of the stem cells and of the cartilage regeneration. In this connection, care has to be taken in particular to ensure that the microfractures do not lie too close to one another, since breaks can otherwise occur between them and compromise the integrity of the bone. If, for example, the surgeon has to perform 10 similar microfractures in an area of one square inch, it needs a high concentration and a great experience to perform an accurate pattern of microfractures.

Therefore, an object of the invention is to provide an instrument with which the microfracturing can be carried out much more easily.

### SUMMARY OF THE INVENTION

According to the invention, the object is achieved by a medical instrument for performing microfractures in a bone, comprising a body having a plurality of points projecting in a distal direction from its distal end.

An instrument is thus made available with which a plurality of microfractures can be created in a single step.

The microfractures are created in a pattern that is precisely defined by the arrangement of the points. In this way, it is possible to effectively avoid breaks between the individually created microfractures.

By means of such an instrument, a microfracturing in a certain area can thus be achieved in a single step. By avoiding breaks between the created microfractures, the integrity of the bone that is to be treated is also guaranteed.

In an embodiment of the invention, the points are distributed uniformly across the distal end.

This measure ensures that, when using the medical instrument, the microfractures are created in the bone at uniform intervals. This leads to a more uniform distribution of the blood and bone marrow emerging from the microfractures and, consequently, of the stem cells contained therein. A more uniform distribution of the stem cells leads in turn to a more uniform regeneration of cartilage tissue and, consequently, to an improved healing of the cartilage defect.

In a further embodiment of the invention, the points project from an end face of the instrument, said points having a length of 3 to 5 mm in particular.

Through the provision of an end face, a limit stop can easily be created which indicates to the operating surgeon that the instrument has penetrated to a sufficient depth into the bone.

On average, in the microfracturing technique, microfractures with a depth of about 4 mm±1 mm are required in order to ensure emergence of blood and bone marrow. Through the combination of an end face, which serves as a limit stop, and points having a length of 3 to 5 mm, it is possible to avoid the points penetrating too deeply into the bone and possibly damaging the bone marrow.

In a further embodiment of the invention, the points are hardened.

Bone is a relatively hard material, which means that signs of wear may appear when a medical instrument of this kind has been used a number of times. Hardening the points reduces these signs of wear, such that the instrument can be used to full effect for a longer period of time. Since the points are also relatively small and thin, breaking-off of a point can be effectively avoided, with the result that no foreign bodies are left in the bone.

In a further embodiment of the invention, the medical instrument further comprises a guide sleeve.

The guide sleeve can be positioned at the operating site by the operating surgeon and serves as a targeting device. The surgeon puts the hollow sleeve on the defect area at the bone and can control, either from the outer side or through the hollow sleeve, the accurate placement. This therefore permits particularly reliable and targeted insertion of the instrument through the hollow sleeve.

By using such a guide sleeve, it is also possible to avoid the bone or surrounding tissue being damaged by the distally projecting points of the instrument during the insertion and positioning of said instrument.

In a further embodiment, the guide sleeve has a punch blade at a distal end.

In the repair of cartilage defects, it is important that all pieces of defective or loose cartilage are removed before the microfracturing is performed. To regenerate the cartilage in a manner free of complications, it is also important that the margins of the healthy cartilage are dissected as smoothly as possible.

If the guide sleeve now has a punch blade at its distal end, the guide sleeve can be used, not only as a targeting device for the instrument, but also as a cartilage puncher.

Such a guide sleeve can be advanced arthroscopically to a cartilage defect and punches the cartilage tissue out around the defect. The tissue that has been punched out in this way can be removed from the guide sleeve with a surgical spoon or a curette, for example. In a next step, the instrument can then be inserted in order to perform the microfracturing.

By means of this combination, a medical instrument can be created with which it is possible to easily and quickly repair a cartilage defect.

In a further embodiment of the invention, the instrument is available in different configurations in terms of its shape, size and the number, arrangement and length of the points.

By means of these measures, a set of instruments can be made available from which an operating surgeon is able to select the instrument suitable for the defect in question. The user-friendliness of the instrument is thus greatly increased.

It will be appreciated that the aforementioned features and those features still to be explained below can be used not only in the respectively cited combination, but also in other combinations or singly, without departing from the scope of the present invention.

### BRIEF DESCRIPTION OF THE DRAWINGS

The invention is described and explained in more detail below on the basis of selected illustrative embodiments and with reference to the attached drawings, in which:

FIG. 1 shows a perspective view of a medical instrument for performing microfractures in a bone,

FIG. 2 shows a perspective view of a guide sleeve for the instrument from FIG. 1,

FIG. 3 shows a highly schematic view, in cross section, of a first step of a first method for performing microfractures in a bone,

FIG. 4 shows a highly schematic view of a second step of the method from FIG. 3,

FIG. 5 shows a highly schematic view of a third step of the method from FIG. 3,

FIG. 6 shows a highly schematic view of a fourth step of the method from FIG. 3,

FIG. 7 shows a highly schematic view of a fifth step of the method from FIG. 3,

FIG. 8 shows a highly schematic view of a sixth step of the method from FIG. 3,

FIG. 9 shows a highly schematic perspective view of a first step of a second method for performing microfractures in a bone,

FIG. 10 shows a highly schematic perspective view of a second step of the second method from FIG. 9,

FIG. 11 shows a highly schematic perspective view of a third step of the second method from FIG. 9,

FIG. 12 shows a highly schematic perspective view of a fourth step of the second method from FIG. 9.

### DETAILED DESCRIPTION OF THE INVENTION

In FIG. 1, an instrument for performing microfracturing in a bone is designated in its entirety by reference number 10.

The instrument 10 has a rod-shaped body 12 with a distal end 14 and a proximal end 16. The cross section of the rod-shaped body 12 is oval.

Since cartilage defects often involve tears, that is to say elongate defects, an elongate cross section is preferred. An oval cross section is preferred because this has no corners that could damage surrounding tissue.

At the proximal end 16, the rod-shaped body 12 widens into a handle 18. A proximal end surface of the handle 18 provides the operating surgeon an application surface for driving the instrument 10 into a bone using his hand or a hammer.

The distal end 14 of the body 12 comprises a plane end face 20. Points 22, here in the form of conical points, project in the distal direction from this plane end face 20. In this embodiment, there are nine points 22, but it is also possible to have a greater or smaller number of points 22.

The points 22 are distributed uniformly across the end face 20. The instrument 10 is made of a metallic material, i.e. titanium.

Microfractures are thus created in a uniformly distributed manner in a bone by means of the instrument 10. This leads to a more uniform distribution of emergent stem cells and to a more uniform healing of a cartilage defect.

The points 22 are hardened with titanium nitride to make them more wear-resistant, and they are used to penetrate a bone in order to create microfractures for the emergence of blood and bone marrow. The length of the points is 5 mm. The nine points are uniformly distributed around the oval face 20 having a longer diameter of about 20 mm and a shorter diameter of about 15 mm.

If these points 22 are now driven into a bone, this can be done until the end face 20 comes to lie on the bone. The end face 20 thus provides a limit stop that prevents the points 22 from penetrating too deeply into the bone.

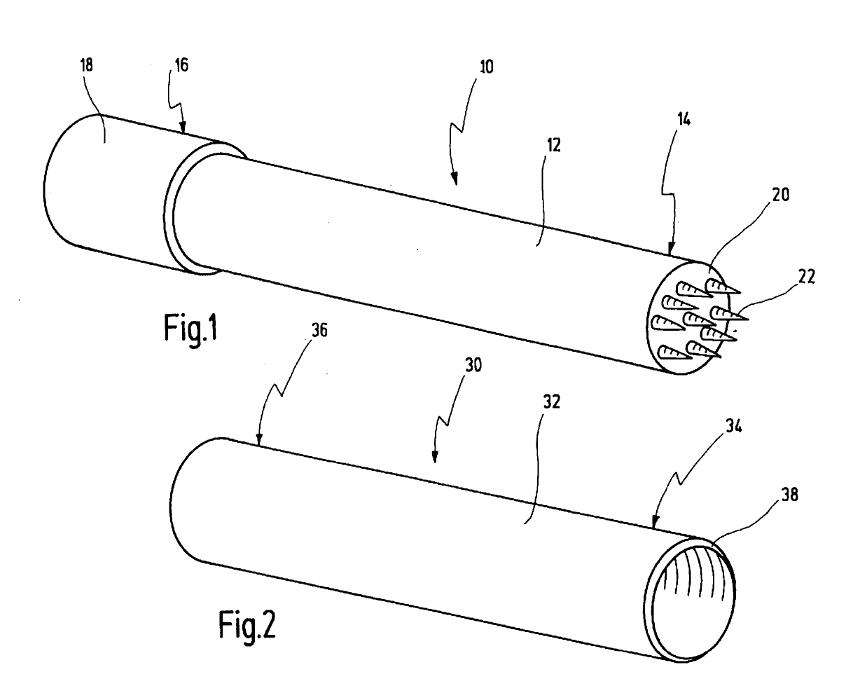

In FIG. 2, a guide sleeve for the instrument 10 from FIG. 1 is designated in its entirety by reference number 30.

The guide sleeve 30 has a tubular body 32 whose inner cross section corresponds to the cross-sectional contour of the body 12 of the instrument 10 from FIG. 1.

The tubular body 32 has a distal end 34 and a proximal end 36. The distal end 34 is ground in such a way that a punch blade 38 is formed that extends round the whole circumference of the tubular body 32. The guide sleeve 30 is also made from titanium.

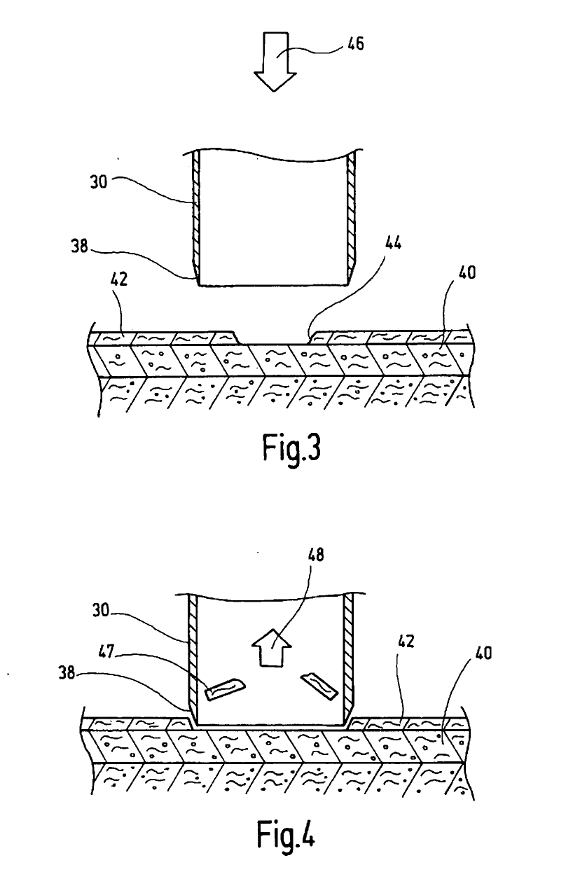

FIG. 3 shows a first step of a method for performing microfractures in a bone.

With arthroscopic visual monitoring, the guide sleeve 30 is advanced to a bone 40 in the knee joint covered by cartilage 42. The cartilage 42 has a cartilage defect 44, which is here in the form of a loss of cartilage tissue.

The bone 40 is shown here with a flat surface. This is done simply for the sake of simplicity. The bone surfaces found in the area of the knee joint are almost exclusively curved bone surfaces.

The arthroscopic instruments used for inserting the guide sleeve 30, for example an arthroscope for visual monitoring, are not shown here.

The punch blade 38 of the guide sleeve 30 is oriented around the cartilage defect 44.

The sleeve 30 is now pressed in the direction of an arrow 46 into the cartilage 42 and moved toward the bone 40, in which process the punch blade 38 punches out the cartilage defect 44.

It will be seen from FIG. 4 that the punch blade 38 of the guide sleeve 30 comes to lie against the bone 40 after the movement indicated by the arrow 46 in FIG. 3. It has now punched out pieces 47 of the cartilage 42. The pieces 47 of the cartilage 42 that have been punched out by the punch blade 38 are now removed from the inside of the guide sleeve 30 in the direction of an arrow 48, with the aid of a surgical spoon (not shown here). Thus, an area of the cartilage 42 has been cut away where the surface of the bone 40 is exposed.

FIG. 5 shows a further step of the method, where the instrument 10 is guided from the proximal direction into the guide sleeve 30.

The instrument 10 is now moved through the guide sleeve 30 to the bone 40 in the direction of an arrow 49.

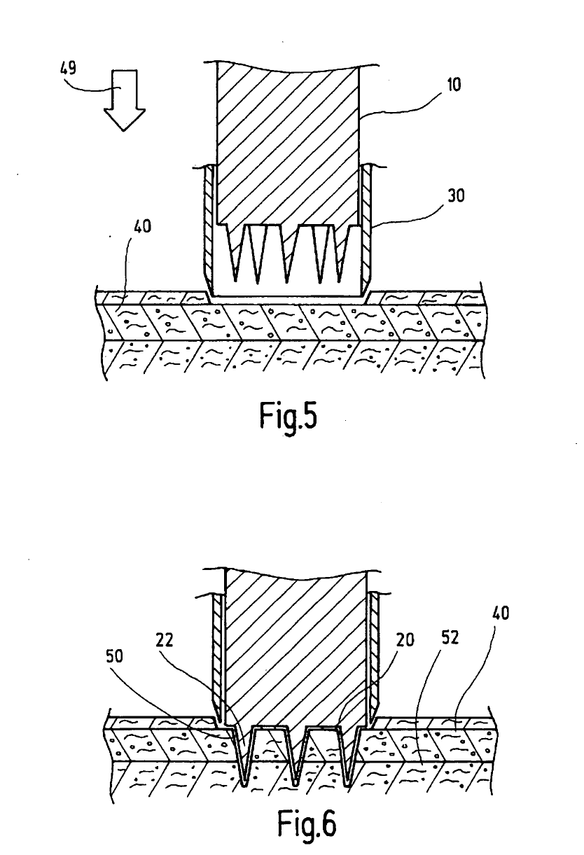

As will be seen from FIG. 6, the points 22 penetrate into the bone 40 until the end face 20 comes to lie against the surface of the bone 40. In this way, microfractures 50 are created in the bone 40. The depth of the microfractures 50 is limited by the end face 20 abutting against the surface of the bone 40. The microfractures 50 extend into bone marrow 52 lying under the bone 40. This bone marrow 52 is particularly rich in pluripotent stem cells.

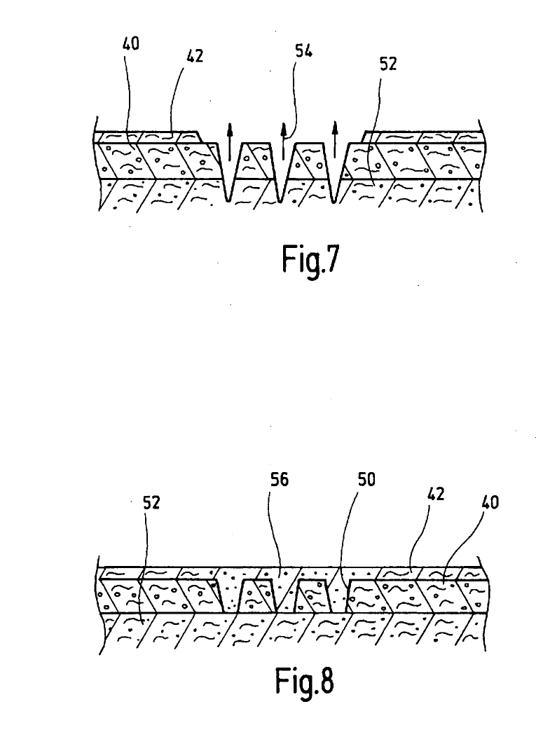

FIG. 7 shows the state of the cartilage 42, bone 40 and bone marrow 52 after removal of the guide sleeve 30 and of the instrument 10.

It will be seen here that the microfractures 50 formed by the points 22 of the instrument 10 remain in the bone 40. Blood, stem cells and other material can now emerge through these microfractures 50 to the surface of the bone 40 in the direction of the arrows 54.

This material forms, on the surface of the bone 40, a clot that is permeated with pluripotent stem cells. These pluripotent stem cells can differentiate to cartilage cells and form new cartilage tissue in the course of the healing process.

FIG. 8 shows the situation after completion of the healing process. The stem cells that have emerged from the bone marrow 52 have differentiated and have formed new cartilage cells 56 in the microfractures 50 of the bone 40 and the cut-out parts of the cartilage 42. In this way, the cartilage defect has been completely rectified.

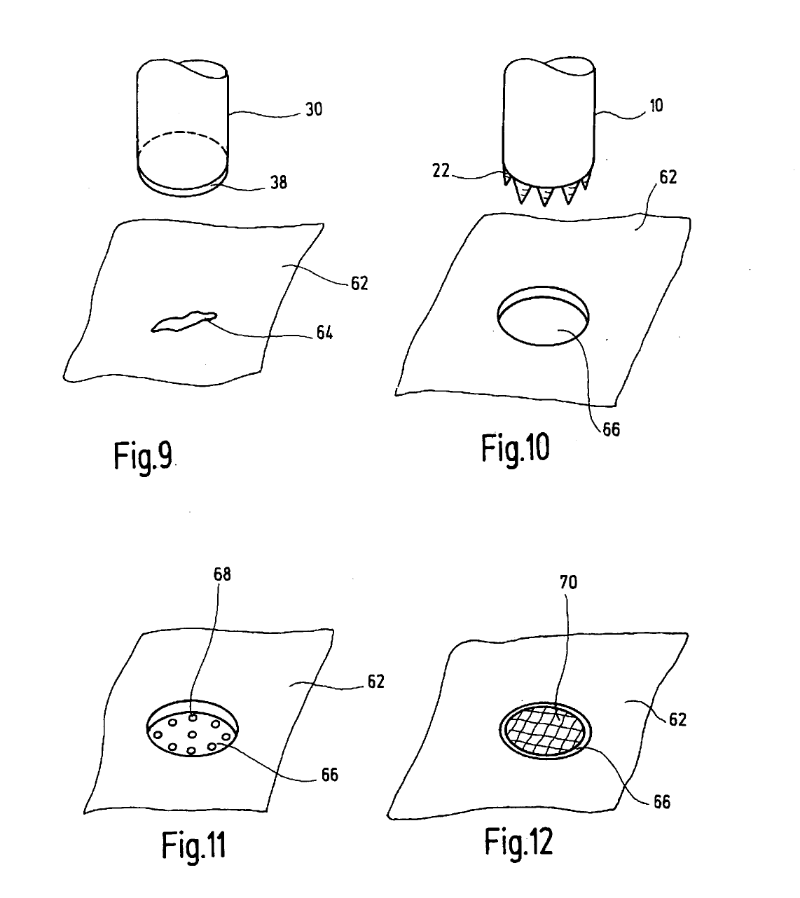

FIG. 9 is a perspective and much simplified representation of a second method for performing microfractures in a bone.

A detail of a cartilage 62 in the knee joint is shown in perspective and comprises a defect 64 in the form of a tear. The guide sleeve 30 is now advanced to the defect by means of arthroscopy until the punch blade 38 comes to lie around the defect 64.

The punch blade 38 is now pressed into the cartilage 62, and the material punched out from the cartilage 62 is removed with a curette after withdrawal of the guide sleeve 30.

For the sake of simplicity, the curette and other known arthroscopic instruments, for example an arthroscope for visual monitoring, that are used in this method have not been shown.

FIG. 10 shows the situation after removal of the cartilage tissue.

A punched-out area 66 is now present in the cartilage 62, and the bone lying under this area 66 is exposed. The instrument 10 is now advanced to the punched-out area. The instrument 10 is then pressed into the punched-out area 66 by the operating surgeon and removed from it again. With an experienced surgeon and with the defect easily accessible, the instrument 10 can be punched in without the aid of a guide sleeve.

FIG. 11 shows the state after the instrument from FIG. 10 has been pressed in and then removed.

It will be seen here that nine microfractures 68 have been formed in a defined pattern in the punched-out area 66.

In a next step, as shown in FIG. 12, a nonwoven 70, with the same size and shape as the punched-out area 66, is now introduced into the punched-out area 66 of the cartilage 62. The pluripotent stem cells emerging from the bone marrow gather in this nonwoven 70. This nonwoven 70 provides the cells with a three-dimensional matrix on which they are able to grow.

The nonwoven 70 itself is composed of pig collagen and degrades in the course of the healing process. This therefore guarantees a particularly effective and targeted growth of new cartilage tissue.

What is claimed is:

1. A medical instrument for performing microfracturing in a bone, comprising a body having a distal end, wherein a plurality of points project in a distal direction from said distal end.

2. The medical instrument of claim 1, wherein said points are distributed uniformly across said distal end.

3. The medical instrument of claim 1, wherein said points project from an end face of said instrument.

4. The medical instrument of claim 1, wherein said points have a length of about 3 to about 5 mm.

5. The medical instrument of claim 1, wherein said points are made of a metallic material and are hardened.

6. The medical instrument of claim 1, wherein said instrument further comprises a guide sleeve for guiding said body therethrough.

7. The medical instrument of claim 6, wherein said guide sleeve has a punch blade at a distal end thereof.

8. The medical instrument of claim 1, wherein said instrument is available in different sizes for performing microfracturing in differently sized areas.

9. The medical instrument of claim 8, wherein said differently sized instrument differs in a number of points projecting from said distal end.

10. The medical instrument of claim 8, wherein said differently sized instrument differs in length of said points.

11. The medical instrument of claim 8, wherein said differently sized instrument differs in a pattern of said points projecting from said distal end.
